(19) United States
(12) Patent Application Publication
(10) Pub. No.: US 2005/0084878 A1
(43) Pub. Date: Apr. 21, 2005

(54) Method for isolating and cloning high molecular weight polynucleotide molecules from the environment

(75) Inventors: Mark R. Liles, Madison, WI (US); Robert M. Goodman, Madison, WI (US); Lynn L. Williamson, Madison, WI (US); Jo E. Handelsman, Madison, WI (US)

Correspondence Address: CROWELL & MORING LLP;INTELLECTUAL PROPERTY GROUP, P.O. BOX 14300, WASHINGTON, DC, 20044-4300, US

(73) Assignee: Wisconsin Alumni Research Foundation, Madison, WI (US)

(21) Appl. No.: 10/846,929

(22) Filed: May 17, 2004

Related U.S. Application Data
(60) Provisional application No. 60/470,899, filed on May 16, 2003.

(51) Int. Cl. C12Q 1/68; C12N 9/42; C12N 1/08

(52) U.S. Cl. 435/6; 435/270

(57) ABSTRACT

Methods and reagents for isolating polynucleotide molecules from uncultured microbial cells in an environmental sample, the method comprising: obtaining uncultured microbial cells from the sample, dispersing the cells from each other and from other components in the sample, purifying the dispersed cells via discontinuous gradient centrifugation wherein the cells are collected in an interface of the gradient, embedding the cells in agarose gel, to produce agarose gel blocks containing the cells, and lysing the cells within the agarose gel blocks, thereby releasing high molecular weight polynucleotide molecules from the cells. Also disclosed are methods and compositions for cloning the polynucleotide molecules so isolated, further comprising incubating the gel blocks with at least a suitable restriction endonuclease to partially digest the polynucleotide molecules, separating the digested polynucleotide molecules by pulse field electrophoresis, to recover fragments of polynucleotide molecules with size of at least 50 kb, ligating recovered fragments of polynucleotide molecules to a suitable cloning vector, and transforming a suitable host cell with the cloning vector containing the fragments of the high molecular weight polynucleotide molecules.

### FEDERAL GOVERNMENT INTEREST

This invention was made with United States government support under a grant from the National Science Foundation (NSF), Grant Number NSF 0132085. The Unites States has certain rights to this invention.

### FIELD OF THE INVENTION

This invention relates to improved methods and compositions useful for isolating and cloning high molecular weight polynucleotide molecules from the environment.

### BACKGROUND OF THE INVENTION

It is now well-established that many, perhaps a vast majority of, microorganisms in certain environmental samples cannot be readily cultivated with known isolation and incubation methods. An alternative to studying the genetic diversity is metagenomics (Handelsman et al., 1998, Chem. Biol. 5:R245-9; Liles et al., 2003, Appl. Environ. Microbiol. 69:2684-2691; Rondon et al., 2000, Appl. Environ. Microbiol. 66:2541-7) where genomic DNAs of two or more unculturable or uncultured microorganisms are directly isolated, cloned and sequenced, screened for genes encoding enzymes for natural product production, or otherwise analyzed (Healy et al., 1995, Appl. Microbiol. Biotech. 43:667-74).

Direct DNA extraction is a fast and simple method where cells in a suspension of the environmental sample (e.g. a soil suspension) are lysed and their DNA extracted, see for example, U.S. Pat. No. 6,261,842, the entire disclosure of which is incorporated herein by reference. This method also provides greater DNA yields (Krsek, et al., 1999, J. Microbiol. Methods 39: 1-16; Miller et al., 1999, Appl. Environ. Microbiol. 65: 4715-4724; Tien et al., 1999, J. Appl. Microbiol. 86: 937-943).

The genomic DNA recovered from lysis of an environmental sample (e.g. soil), however, may be derived from non-microbial sources. Furthermore, direct DNA extraction results in DNA of less than 100 kb in size, and often containing substantial contaminants such as humic substances that interfere withsubsequent manipulation of the DNA.

There is a significant advantage in the isolation of high molecular weight (HMW) genomic DNA for the construction of large-insert genomic libraries. Cloning large genomic DNA fragments increases the probability that a clone will contain all of the genes encoding a biosynthetic pathway, or that a clone containing a phylogenetic marker (e.g., 16S rRNA gene) will also contain other functional genes of interest (Rondon et al., 2000, Appl. Environ. Microbiol. 66:2541-2547; Liles et al., 2003, Appl. Environ. Microbiol. 69:2684-2691).

Therefore, there is a need for methods with which one can efficiently isolate high molecular weight polynucleotide molecules that are substantially free of contaminants and are suitable for cloning and metagenomics studies.

### SUMMARY OF THE INVENTION

The invention generally provides methods and reagents for culture-independent isolation of polynucleotides from microbial cells in an environmental sample, the method comprising: obtaining microbial cells from the sample, dispersing the cells from each other and from other components in the sample, purifying the dispersed cells via discontinuous gradient centrifugation wherein the cells are collected in an interface of the gradient, embedding the cells in agarose gel, to produce agarose gel blocks containing the cells, and lysing the cells within the agarose gel blocks, thereby releasing high molecular weight polynucleotide molecules from the cells. In a preferred embodiment, sodium metaphosphate is included in the suspension prepared from the sample to increase the yield of the polynucleotides recovered from the sample.

Also disclosed are methods and compositions for cloning the polynucleotide molecules so isolated, the method further comprising incubating the gel blocks with at least a suitable restriction endonuclease to partially digest the polynucleotide molecules, separating the digested polynucleotide molecules by pulse field electrophoresis, to recover fragments of polynucleotide molecules with size of at least 50 kb, (optional step of removing agarose gel), ligating recovered fragments of polynucleotide molecules to a suitable cloning vector, and transforming a suitable host cell with the cloning vector containing the fragments of the high molecular weight polynucleotide molecules.

A further embodiment of the invention provides a metagenomic library comprising host cells comprising clones so produced.

### BRIEF DESCRIPTION OF THE DRAWINGS

FIG. 1 is a diagram of steps involved in isolation of HMW genomic DNA from soil microflora.

FIG. 2 is a pulsed field agarose gel showing a comparison between genomic DNA isolated by 1) direct extraction and 2) indirect extraction. Each lane contains the amount of genomic DNA isolated from approx. 1 g of soil. Molecular weight sizes are based upon yeast chromosomal PFG (pulsed field gel) electrophoresis markers.

FIG. 3 shows the insert size distribution of clones in metagenomic libraries constructed from BCEF soil microorganisms via direct extraction (AK1-4), via indirect extraction (AK5), and via indirect extraction plus size-selection (AK7).

FIG. 4 shows that the restriction endonuclease Sau3AI and Clean-Cut™ agarose achieves efficient partial digestion of HMW DNA isolated according to a method of the present invention.

### DETAILED DESCRIPTION OF PREFERRED EMBODIMENTS

As discussed above, direct DNA isolation from environmental samples has many drawbacks. Indirect DNA extraction involves an additional step where microbial cells are first isolated from the environmental sample, followed by cell lysis, and DNA extraction and purification.

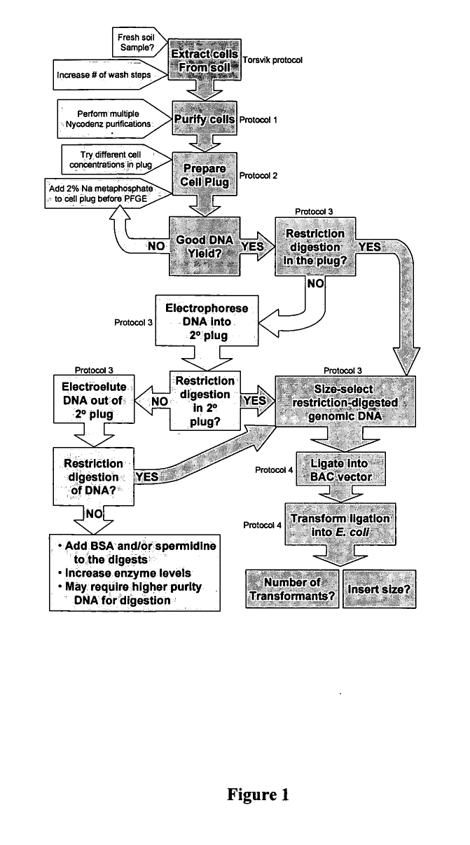

The indirect approach helps to maintain the integrity of genomic DNA during the isolation process. Microbial cells are first separated from soil or other environmental samples, and embedded in an agarose gel block or plug, after which the cells are lysed in the agarose matrix (FIG. 1). The isolation of DNA from microbial cells that have been separated from environmental samples such as soil matrix results in polynucleotide molecules that are significantly larger and less contaminated with humic materials than DNA recovered by methods involving lysis of microbes in the soil matrix, without prior separation (Bakken and Lindahl, 1995, Recovery of bacterial cells from soil. In: van Elsas and Trevors (Eds.), Nucleic Acids in the Environment: Methods and Applications. Springer Verlag, Berlin: pp. 9-27); Faegri et al., 1977, Soil Biol. Biochem. 9:105-112; Herron et al., 1990, Appl. Environ. Microbiol. 56:1406-1412; Lindahl, et al., 1995, Fems Microb. Ecol. 16:135-142; Steffan et al., 1988, Appl. Environ. Microbiol. 54:2908-2915; Tebbe et al., 1993, Appl. Environ. Microbiol. 59: 2657-2665). Humic materials are known to interfere with subsequent DNA manipulation processes (e.g. purification, digestion or ligation for cloning purposes).

One approach to isolating microbial cells from the samples without culturing is through gradient centrifugation. Previous attempts, however, were unsuccessful because environmental samples often contain particles or components that are similar to the microbial cells in physico-chemical properties and render separation of cells from such particles or components ineffective.

For example, the present inventors attempted to isolate HMW DNAs from various soil samples using prior art methods (Berry et al., 2003, FEMS Microbiol. Lett. 223:15-20), but were met with only limited success with one soil type from the Hancock Agricultural Research Station of the University of Wisconsin, a particularly sandy soil with low organic content. With all other soil types tested (n=4), when gradient centrifugation was employed, all the cells were pelleted at the bottom of the gradient, likely due to the adsorption of cells onto soil particulates (e.g., clay particles) that sedimented to the bottom of the gradient tube. Therefore, it was not possible to isolate microbial cells using prior art methods. With these soil types, two of which are typical of Midwestern prairie soils, the inventors attempted to isolate DNA from the recovered cells without conducting cell purification via gradient centrifugation. Despite using multiple methods to remove humic and other contaminants from the extracted (but not gradient purified) bacterial cells, cell lysis was never achieved using enzymatic treatments. Although the embedded cells from organic-rich soils could be lysed when subjected to boiling in the presence of 1% sodium dodecylsulfate (SDS), the recovered DNA was less than 50 kb in size. The inventors concluded from this research that gradient centrifugation was necessary to isolate HMW DNA from microbial cells isolated from soil, but that the prior art methods were insufficient to allow this from most common soil types.

The present inventors now surprisingly discovered that by combining a dispersion step, whereby microbial cells are separated from each other and from other particles in the sample, and a step of gradient centrifugation, preferably using a specific gradient forming material, microbial cells from environmental samples can be effectively recovered and their DNA isolated with improved purity while retaining their high molecular weight characteristics.

As used herein, the term “microorganism” includes prokaryotic and eukaryotic microbial species from the Domains Archaea, Bacteria and Eucarya, the latter including yeast and filamentous fingi, protozoa, algae, or higher Protista. The terms “microbial cells” and “microbes” are used interchangeably with the term microorganism.

As used herein, the term “polynucleotides” or “nucleic acids” refers to deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. The term “kb” refers to kilobases, i.e., a thousand contiguous nucleotide bases in a single, double-stranded nucleic acid molecule, or kilobase pairs in a double stranded nucleic acid molecule. The term “Mb” refers to megabases, i.e., a million contiguous nucleotide bases in a single nucleic acid molecule, or megabase pairs in a double-stranded nucleic acid molecule.

In a preferred embodiment, the microbial cells are separated and isolated from the environmental samples using density gradient centrifugation. Techniques of density gradient centrifugation are well known in the art. Suitable density gradient centrifugation media are those where the additive forms a solution in water within the required density range, does not interfere with, or damage, the microbial cells in the sample, and the solution has a refractive index within the practical range, as well as a low viscosity. In addition, the additive must be easily removable from the sample.

Many materials are available for forming a density gradient suitable for the present invention. Examples include salts of alkali metals (such as cesium chloride, sodium iodide, sodium bromide, cesium sulfate, cesium acetate, potassium tartrate), neutral, water-soluble molecules (such as glucose, sucrose glycerol), hydrophilic macromolecules (such as dextran), synthetic molecules (e.g. sodium or methyl glucamine salt of triiodobenzoic acid and of metrizoic acid, and metrizamide.

For the purpose of the present invention, a discontinuous density gradient centrifugation is preferred. Suitable ingredients for preparing discontinuous density gradients include Nycodenz™ (5-(N-2,3-dihydroxypropylacetamido)-2,4,6-triiodo-N,N′-bis(2,3-dihyroxypropyl)sophthalamide, Iodixanol® (5,5′-[(2-hydroxy-1-3-propanediyl)-bis(acetylamino))bis[N,N′bis(2,3-dihydroxypropyl)-2,4,6-triiodo-1,3-benzenecarboxamide]), and Percoll® (available from Pharmacia, Piscataway, N.J.). It is readily recognized that the density of the centrifugation gradient should be adjusted such that the cells to be isolated are collected, after a suitable centrifugation, in the interface between the two phases of the gradient, while the other components of the environmental sample will be either at the bottom of the gradient or at the top of the gradient.

Nycodenz® density gradient centrifugation is particularly preferred as a further purification method (Courtois, et al., 2003, Appl. Environ. Microbiol. 69: 49-55), in order to acquire bacterial cells that can be lysed within agarose.

Without use of dispersal agents (see below), the vast majority of the extracted cells from the organic-rich soil penetrated the Nycodenz® layer, ending up in the pellet after centrifugation. Based on the results of MacDonald and others (Jacobsen et al., 1992, Appl. Environ. Microbiol. 58: 2458-2462; Macdonald, 1986, Soil Biol. Biochem. 18:399-406; Macdonald, 1986, Soil Biol. Biochem. 18:407-410; Macdonald, 1986, Soil Biol. Biochem 18:411-416; Turpin et al., 1993, J. Appl. Bacteriol. 74:181-190), we hypothesized that small clay particles are likely responsible for bringing the cells into the Nycodenz layer.

To solve this problem, a combination-of enzymatic and resin treatments was used to disperse the bacterial cells from the small clay particles.

According to a preferred embodiment of the invention, microbial cells are separated or dispersed from other components of the environmental sample, such as mineral or organic particles in a soil sample. Also, microbial cells should be separated from each other, so that they can be separated from the other components in the environmental sample more effectively via density gradient centrifugation.

Such separation or dispersion may be achieved by many methods. For example, cells can be dispersed by the use of a suitable surfactant such as SDS, a suitable solution or buffer, an enzyme, a suitable macromolecule or resin, or a combination thereof. Preferably, the cells are dispersed with a combination of sodium deoxycholate, polyethylene glycol (PEG) and an ion exchange resin, especially an anion exchange resin such as Chelex-100.

Another method of separating microbial cells from clay particles is a physical means such as low energy sonication or homogenization.

The cells in the gradient form a band after appropriate centrifugation. The cells are collected, further washed one or more times, and prepared into a cell pellet or suspension for embedding and subsequent processing.

The isolated cells are then embedded in a suitable agarose gel, which are then cut into plugs or small blocks. The bacterial cells are lysed within an agarose plug by chemical and enzymatic means, and the HMW DNA visualized and recovered from a pulsed field agarose gel. Enzymatic lysis will not be suitable for all bacterial groups, especially spore-formers such as the Bacilli, in which case harsher methods may need to be employed to acquire their genomic DNA (Duarte et al., 1998, J. Microbiol. Methods 32: 21-29; Kozdroj et al., 2000, Biol. Fertil. Soils 31: 372-378).

It is also known that components of environmental samples, e.g. clay particles, adsorb DNA, thus decreasing yield of the nucleic acid molecules of the isolation procedure. The present inventors have surprisingly discovered that sodium metaphosphate binds to these DNA attachment sites in the clay particles, thus preventing the loss of DNA during the isolating process. Incubating the cell-embedded agarose plugs in 2% sodium metaphosphate (ph 8.5) for about 10 hours or more at 4° C. is sufficient to increase the DNA yield more than 5-fold for some soil samples, especially soils with high clay contents. The inventors also found that adding denatured salmon sperm (1 mM) to the agarose plugs increases the DNA yield, demonstrating another method of saturating any DNA attachment sites on clay or other particles that were co-isolated with the cells.

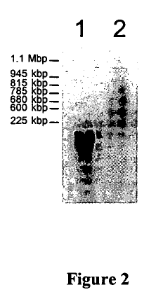

The agarose blocks or plugs, now containing the purified NMW DNA are subjected to pulse field gel electrophoresis. The genomic DNA recovered from the pulsed field gel using methods of the present invention is significantly larger than genomic DNA recovered by direct lysis (see FIG. 2), and is sufficiently pure for restriction digestion and BAC or cosmid cloning.

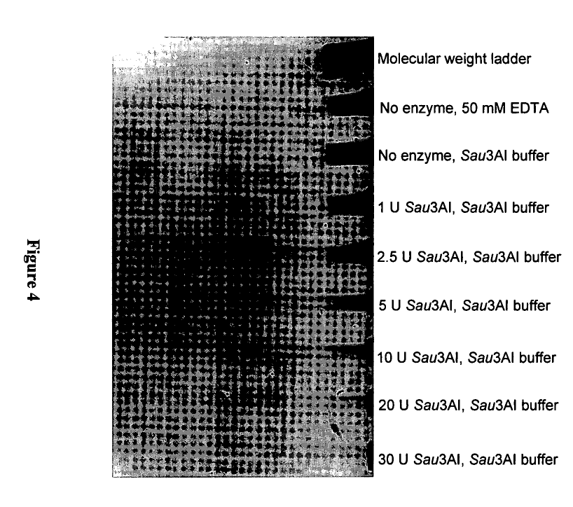

After the electrophoresis, the DNAs are subsequently isolated from appropriate portions of the gel, and are partially restriction digested with a suitable restriction endonuclease for cloning into a suitable vector. Again, to minimize DNA shearing, digestion is preferably done while the DNA remains inside the gel plugs or blocks. Methods of the present invention achieves efficient and satisfactory restriction digestion. With the restriction endonuclease Sau3AI and Clean-Cut™ agarose (available from BioRad), more than 10-fold improvement in digestion compared to the prior art method was achieved. Furthermore, in some soils there is evidence of DNA nuclease activity after cell lysis, which can impair cloning efficiency (See FIG. 4). To circumbvent this problem, the inventors conduct restriction digests at 4° C., for 5-10 hours, thereby allowing restriction digestion with the Sau3AI restriction endonuclease at the lower temperature, but minimizing any non-specific nuclease activity that may be present in the agarose plugs.

In a preferred embodiment, the partially restriction digested HMW DNA is electroeluted within a dialysis membrane following standard molecular biology protocols well-known to those in the art. This achieves improved recovery of DNA compared to electroelution of the DNA into a well cut in the gel. Preferably, the DNA in the dialysis membrane may be concentrated using polyethylene glycol, and dialyzed using a buffer, e.g. a buffer containing 10 mM Tris, 1 mM EDTA, pH 8.0.

In a preferred embodiment, the isolated polynucleotides are further cloned into a suitable host cell using a suitable vector. Methods of cloning isolated and suitably restriction digested HMW DNA are known in the art, and involve ligating the HMW DNA to be cloned to a suitable vector and transforming the ligated vector into a suitable host cell.

The terms “host cells” and “recombinant host cells” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Representative examples of vectors which may be used include viral vectors, phage, plasmids, phagemids, cosmids, phosmids, bacterial artificial chromosomes (BACs), bacteriophage P1, P1-based artificial chromosomes (PACs), yeast artificial chromosomes (YACs), yeast plasmids, and any other vectors suitable for a specific host cell and capable of stably maintaining and expressing a genomic DNA insert of at least 20 kb, and more preferably greater than 50-75 kb.

Preferred vectors for the present invention are the so-called artificial chromosomes. One feature of these vectors is their ability to carry large genetic inserts, e.g., greater than 50 kb and up to 350 kb. The low copy number of the bacterial artificial chromosome (BAC) vector, (i.e., one copy of DNA per each host cell) provides a high degree of stability of these vectors in a restriction and recombination-deficient E. coli host. The upper limit on the size of the insert is often big enough that hundreds of genes can be included on one vector. The utility of the BAC system in large-scale genomic mapping efforts has led to the development of protocols optimized specifically for these plasmids with large inserts (Birren et al., 1993, in Pulsed Field Gel Electrophoresis. Academic Press, San Diego; Sheng et al., 1995, Nucl. Acids Res. 23:1990-1996; and Wang et al., 1995, Electrophoresis 16:1-7).

In one embodiment, ligation of the HMW isolated and prepared according to methods of the present invention, into e.g. BAC vectors, is without using shrimp alkaline phosphatase. This decreases the number of background colonies without insert. Ligation products can be directly electroporated into electrocompetent E. coli cells.

The method of the present invention is applicable to many types of environmental samples. For example, bacteria and fungi that live in association with plants, either as endophytes (within the plant) or on the plant rhizosphere (in close proximity to the root surface), may be distinct from those microorganisms living in bulk soil. Furthermore, these plant-associated microorganisms may have important roles in promoting plant health or disease processes. A further embodiment of this method would be to isolate the soil adhering to roots (e.g., rhizosphere) and only isolate DNA from the root-associated microorganisms. A collection of DNA from rhizosphere soil would be considered to be enriched for plant root-associated microorganisms, and would be predicted to contain genes involved in harvesting energy from plant root exudates, and in promoting plant health through mobilization of trace elements and other plant growth-stimulating compounds.

### EXAMPLES

### Example 1

### Extraction of Bacterial Cells From Soil

1. Weigh out 4-6 portions of 30 g soil, previously sieved through a soil sieve, mesh size 2 mm. Homogenize each portion with 100 ml ice-cold sterile distilled water in a Waring blender at low speed three times for 1 min, with 5 min cooling on ice (or 1 min in a freezer) between each run.

2. Transfer the soil suspensions to 500 ml centrifuge flasks with 300 ml sterile water, and centrifuge for 15 min at 5° C. and 1,000×g.

3. Pool the supernatants and store cold (4° C.) until further processing. Transfer the soil pellets back into the blender with 100 ml sterile water and homogenize anew for 1 min. Then, transfer the homogenate to the centrifuge flasks with 300 ml sterile water and centrifuge as before. The supernatants are combined with the earlier pooled supernatants. The procedure is repeated once.

4. Centrifuge the combined supernatants from the three low-speed centrifugations batch-wise for 30 min at high speed (10,000×g) and 5° C. (e.g., 9,000 r.p.m. in a Sorvall RC-5 centrifuge with GSA rotor). Discard the supernatants from these runs, and leave the pellets in the flasks until the total volume has been centrifuged.

5. Combine the bacterial pellets and re-suspend them in 200 ml cold 2% (w/v) sodium metaphosphate, adjusted to pH 8.5 with 0.2% (w/v) Na₂CO₃. Homogenize in the Waring blendor for 1 min at low speed and centrifuge at 10,000×g and 5° C. for 30 min.

6. Resuspend the pellet in 200 ml Crombach buffer and centrifuge as before. Transfer the pellet to a 30 ml centrifuge tube with 20 ml Crombach buffer and centrifuge again (SS-34 rotor) at 10,000×g and 5° C. for 30 min.

The procedure described in Example 1 is an adaptation of that described in Torsvik, V., 1995, In: Akkermans et al. (Eds), Molecular Microbial Ecology Manual, sect. 1.3.1. Note that there is no cell fixation step using isopropanol, which could prevent cell-particle dispersion and reduce the effectiveness of the Nycodenz gradient purification step below.

Additional notes: In step 1, add 10 g per liter PVPP or PVP as suggested by Torsvik. Steps 5 and 6 can be repeated if the supernatant is dark after the 15,000×g spin, until the supernatant is relatively clear (usually repeat 2 to 3 times). Alternating washes in metaphosphate buffer followed by Crombach buffer was effective, although the pellet was less stable after the Crombach wash. To resuspend the pellet, scraping with a sterile spatula or other device is more preferable to pipetting. Then the clumps of material are transferred into a blender for homogenization.

### Example 2

### Dispersal and Purification of Bacterial Cells

Prior to using Nycodenz density gradient centrifugation as a further purification method, a combination of enzymatic and resin treatments was used to disperse the bacterial cells from the small clay particles that are likely responsible for bringing the cells into the Nycodenz layer.

The specific used in this examples are:

1. Suspend the bacterial cell pellet after step 6 in Example 1 in 40 ml dispersion solution (2% sodium hexametaphosphate buffer, pH 8.5 containing 0.2% (w/v) sodium deoxycholate, 25 mg ml⁻¹ polyethylene glycol, and 20 mg ml⁻¹ Chelex-100 resin (pH equilibrated; Sigma) by removing the pellet from the sides of the tube with a sterile spatula or by vigorous pipetting.

2. Incubate the fine suspension in a 250 ml bottle at 4° C. for 2 hours, with gentle mixing by placing the bottle on a rotating platform at 100 rpm.

3. Pipet 20 ml of cell suspension into two Round-bottom, clear 50 ml centrifuge tubes (we use Nalgene #3117-0500, ordered from Fisher), and then underlay the cell suspension with 7 ml of a Nycodenz solution (1.3 g per ml of distilled water, autoclaved for 30 min, and cooled to 4° C.) using a Pasteur pipet. Be careful not to carry over any resin to the Nycodenz-containing tubes. Add sufficient dispersion solution to fill each ultracentrifuge tube.

4. Incubate the tubes in a 4° C. cold room for 30 min to allow larger particles to settle to the bottom of the suspension prior to centrifugation.

5. Subject the tubes to 20 min of centrifugation at 10,000×g at 4° C. in a SW28 swinging bucket rotor. A band containing bacterial cells will be resolved at the Nycodenz-aqueous interface.

6. Aspirate the sodium metaphosphate buffer comprising the upper layer to within 2 to 3 mm of the Nycodenz interface, and replace with approximately 10 ml chilled 2% metaphosphate buffer. Gently swirl each tube for 1 min, while intermittently gently pipetting the cells off of the Nycodenz cushion. Remove the upper buffer layer to a sterile 30 ml tube.

7. Add metaphosphate buffer to bring each tube to 30 ml total, thoroughly mix by gentle inversion 4-6 times, and pellet the cells by centrifugation at 10,000×g for 15 min at 4° C.

8. Wash the cell pellets in 1 ml chilled STE buffer at least twice, and pellet the cells in a microcentrifuge tube subjected to 10,000×g for 3 min each time.

9. Resuspend the cells in 500 μl STE buffer, and subject to 3 rounds of sonication for 15 sec each at 15V, in order to produce a fine cell suspension.

In step one, we also have used low-energy sonication to achieve a fine suspension. A probe sonicator (Sonics Vibra Cell 250) set at 38 V (or equivalent low-energy setting) was used for 3 bursts of 15 sec each, while cooling on ice. The Chelex-100 resin is added to 500 ml sterile H₂O, and the pH adjusted to 8.5 prior to using the resin in the dispersion solution.

We have also used AG 501-X8 resin, which is a mixture of anion and cation resins. This resin also induces cell-particle dispersal similar to that achieved with Chelex-100.

We used very low-energy sonication to achieve a fine suspension in order for the enzymatic and resin treatment to be effective. This sonication level is sufficiently low as to avoid cell lysis. As an alternative to sonication, homogenization with a tissue homogenizer may be more effective with some soils.

With some soil types (e.g., silty soils) we found it more efficient to pipet the cell layer directly off the Nycodenz cushion without resuspending the cells. We also found that performing sequential Nycodenz purifications dramatically decreased cell yield, but the resultant doubly or triply purified cells were readily lysed.

For efficient cell lysis within an agarose plug, it is very important to produce a fine cell suspension at this step, immediately prior to embedding. This may also be achieved with a tissue homogenizer.

Equipment needed for the above procedure include probe sonicator (e.g. Sonics Vibra Cell 250 or equivalent) or tissue homogenizer, and an ultracentrifuge (e.g. Beckman L8-80M or equivalent). Chemical reagents and solutions include Crombach buffer (0.033 M Tris hydrochloride, 0.001 M sodium ethylene diamine tetra-acetate (EDTA), pH 8.0); a dispersion solution (2% sodium hexametaphosphate buffer, pH 8.5 containing 0.2% (w/v) sodium deoxycholate, 25 mg ml⁻¹ polyethylene glycol, and 20 mg ml⁻¹ Chelex-100 resin (pH equilibrated; Sigma)), Nycodenz solution: 1.3 mg per ml (5-(N-2,3-Dihydropropylacetamido)-2,4,6-Trilodo-N,N′-bis(bis(2,3-Dihydroxypropyl)-isophtalamide), Sigma#D-2158, autoclaved and cooled to 4° C., and STE buffer (1 M NaCl, 0.1 M EDTA, 10 mM pH 8.0).

### Example 3

### Embedding Cells Within Agarose and Cell Lysis

Once a purified cell suspension has been achieved, bacterial cells may be lysed within an agarose plug by chemical and enzymatic means, and the HMW DNA visualized and recovered from a pulsed field agarose gel. Researchers targeting specific groups by this method could use group-specific rRNA-targeted primer sets to ascertain whether the DNA recovered includes the intended group(s).

Enzymatic lysis, however, may not be suitable for all bacterial groups, especially spore-formers such as the Bacilli, in which case harsher methods may need to be employed to acquire their genomic DNA (Duarte et al., 1998, J. Microbiol. Methods 32: 21-29; Kozdroj and van Elsas, 2000, Biol. Fertil. Soils 31: 372-378).

The steps in the procedure of this example are:

1. Prepare a 1.4% low melting point agarose (Promega) solution in 1×TAE, and cool to 45° C.

2. Rapidly mix 500 μl of the agarose solution with an equal volume of suspended, purified cells (Protocol #2, step 9), and take up the entire solution into a 1 cc syringe. Place the cap on the syringe, and allow to cool at 4° C. until set, approximately 30 min.

3. Extrude the cell-agarose “worm” from the syringe (the end of the syringe may need to be cut off with a clean razor blade) into the lysis buffer contained within a 50 ml centrifuge tube.

4. Add 10 ml of lysis buffer to these plugs, with 1 ml solution per each plug (this assumes each plug is approx. 100 μl. As a general guide, use 10× volume of the plug for all incubations).

5. Incubate for 3 hours at 37° C., with gentle agitation (we use a 50 ml conical centrifuge tube placed inside a Techne hybridization oven, which gently rotates the tube, for steps 5 through 10).

6. Remove the lysis buffer, and replace with 40 ml of ESP buffer, and incubate at 55° C. for 16 hours (overnight), with gentle agitation.

7. Remove the ESP solution, and replace with fresh ESP solution for another hour at 55° C., with gentle agitation.

8. Remove ESP solution, and wash 3 times in 1 ml TE per plug at room temperature for 10 min each time.

9. Incubate plugs two times in 1 ml TE per plug plus 1 mM PMSF (a protease inhibitor). Let plugs incubate at room temperature for an hour each time. Make up a 100 mM PMSF stock in isopropanol, and be careful, as PMSF is very toxic. Discard buffer containing PMSF in special bottle.

10. Wash plugs twice in 1 ml TE per plug at room temperature for 10 min each time.

11. Incubate plugs overnight in storage buffer at 4° C.

12. Lysed plugs are now ready for use, and are stable at 4° C. in storage buffer for extended periods (at least weeks, and probably months). Be certain to wash the plugs in TE to remove the extra EDTA in the storage buffer before the subsequent step(s).

13. To evaluate DNA yield and size, place one plug (or a slice of a plug) within a well of a 1% agarose gel, and subject the DNA to pulsed field electrophoresis at 6 V/cm for 10 hours at 14° C. with a 3 sec to 15 sec switch time and a 120° angle.

Equipment needed include sterile 1 cc syringes; Hybridization oven with rotating cylinders (or equivalent). Chemicals, reagents, solutions include lysis buffer (10 mM Tris, 50 mM NaCl, 0.2 M EDTA, 1% sarkosyl, 0.2% sodium deoxycholate, 1 mg ml⁻¹ lysozyme, pH 8.0); ESP buffer (1% sarkosyl, 1 mg ml⁻¹ proteinase K (1.4 U mg⁻¹, 0.5 M EDTA, pH 8.0); storage buffer (10 mM Tris-HCl, 50 mM EDTA, pH 8.0).

Cells may be stored in the syringe at 4° C. for several weeks. We have tried using different concentrations of cells within an agarose plug, and found that there is a direct relationship with numbers of cells embedded in agarose and DNA yield from the plugs, with no loss of yield with high cell concentrations (˜10¹⁰-10¹¹ ml⁻¹). This may vary with soil type, so it advisable to at first try a range of cell concentrations in agarose plugs, to establish optimal DNA yield.

We have used plugs stored for 2 months in storage buffer, with no apparent loss of DNA yield, or nuclease activity. The stability of genomic DNA within agarose plugs may vary among soils. In our experience the stability of genomic DNA is considerably reduced once the genomic DNA has been electrophoresed into an agarose gel.

### Example 4

### BAC Cloning

Efficient restriction digestion of HMW DNA embedded within agarose plugs is achieved by two means: 1) electrophoresing the DNA into clean-cut agarose (BioRad), 2) using the restriction endonuclease Sau3AI, and 3) performing restriction digestion at 4° C. Our first attempts at cloning genomic DNA involved electrophoresing HMW genomic DNA into an agarose (standard agarose) gel, and then isolating a compressed band of genomic DNA greater than 300 kb in size from the gel. We refer to this agarose plug containing the HMW DNA as a secondary (2°) plug. The isolated HMW DNA was then digested with various restriction endonucleases. Unfortunately, we found that substantial nuclease activity was present in some samples that did not have restriction enzymes added, resulting in the degradation of genomic DNA present in the HMW DNA plug. While the genomic DNA appears very stable while it is in the primary plug, genomic DNA stability is very poor once it has been electrophoresed from the first plug. Therefore, the following method minimizes non-specific genomic DNA degradation by performing the restriction digestion at 4° C., and improves the efficiency of restriction digestion by electrophoresing the HMW DNA into clean-cut agarose and using the endonuclease Sau3AI, which in our experience is the only restriction enzyme capable of digesting soil microbial genomic DNA within a cell plug. Once the restriction digestion has occurred, we rapidly perform the next several steps, from size-selection to cloning, to minimize further degradation. The efficiency of each step subsequent to digestion is dramatically decreased with storage time, and even maintaining samples at 4° C. with EDTA does not prevent the decline of cloning efficiency. By rapidly isolating and ligating size-selected, restriction-digested genomic DNA into a BAC vector, this problem may be overcome, and large-insert genomic libraries produced. The following protocol has worked with several different soil types, but many of the conditions (i.e., restriction digestion, number of cell plugs used) must be determined empirically for each environmental sample.

Steps in the procedure:

A. Partial Restriction Digest

Incubate plugs in 1 ml TE per plug for 30 min if they are in storage buffer

Incubate a cell plug in 1×Sau3A I buffer “B” (Promega) with 1× polyamines and 1×BSA for 30 min at 4° C.

Remove buffer, and replace with fresh pre-chilled buffer with 10-50 units Sau3A per 300 ul (the number of enzyme units will vary considerably with each soil type and degree of DNA purity)

Incubate at 4° C. (on ice) for 5-10 hours

Add 500 microliters of chilled storage buffer (50 mM EDTA)

Wash two times in storage buffer on ice, 10 min each, and leave on ice until the gel is ready.

B. Size-Selection via Pulsed Field Gel Electrophoresis

Prepare 1% agarose gel in 1×TAE (we have used both Seaplaque GTG and Promega low-melting point (LMP) agarose, to equal effect).

Cut the lanes of the gel in which the cell plugs will be placed with an EtOH-washed razor blade, so that the lane extends from the edge of the gel to the normal lane position.

Place cell plugs along the entire length of the gel.

Fill in the remaining portion of the lane with molten, cooled (˜45° C.) 1% agarose

Immediately when the gel solidifies, place within a PFGE apparatus, with the 1×TAE buffer pre-chilled to 14° C.

Add molecular weight markers (we use NEB PFGE mid-range marker II)

Run pulsed-field gel at 5 v/cm for 10 h at 14° C., with 3 to 30 sec switch time, and linear ramping

Stain a portion of the gel including MW markers and a ˜5 mm section of your cell plug lane with EtBr.

Remove the section of the gel corresponding to the 50 (or 100) kb+region, using a clean, EtOH-washed razor blade. Minimize the volume of the agarose slice.

C. Ligation

Place the size-selected agarose section within a SpectraPor 2 dialysis membrane (boiled for 10 min, washed in 1×TAE). Remove all 1×TAE buffer possible, and seal the dialysis membrane with clips. Enough buffer will remain in the membrane for DNA to electrophoresis into the buffer. Place the dialysis membrane into an electrophoresis chamber with pre-chilled 1×TAE buffer, and electrophorese at 70 V for 3 hours (we place the chamber in a cold room during the electrophoresis). After 3 hours, reverse the polarity on the electrophoresis chamber, and electrophorese for 1 min at 70V to prevent DNA from attaching to the dialysis membrane.

Carefully and slowly remove the retained buffer from within the dialysis membrane and place in a microcentrifuge tube on ice.

Quantify DNA concentration by running 5-50 μl of the electroeluted DNA sample on a 1% agarose gel, with lambda quantification standards.

Add 10-88 μl of the insert to 25 ng BamHI-digested, phosphatased BAC vector (in 1-10 μl volume) using a wide-bore 200 μl pipet tip. Heat at 55° C. for 5 min (optional), then place on ice for 5 min.

Add 10 μl 10× T4 DNA ligase buffer (after making sure that the ATP is in solution by vigorous vortexing), and 5 units T4 DNA ligase (Promega). Pipet the solution gently with a wide-bore pipet tip 10 times, and incubate the ligation for 16 hours (overnight) at 15° C.

D. Transformation

Remove salts present in the ligation buffer by either agarose cone dialysis, or membrane filter dialysis (we use Millipore Type VS, 0.025 μm) against ddH₂O. Place the 100 μl ligation reaction into the agarose cone, or onto the membrane, and allow dialysis to proceed for 1 hour.

Remove the dialyzed ligation products using a wide-bore pipet tip into a 1.5 ml microcentrifuge tube, and place on ice.

Chill electrocompetent E. coli cells (we use either Epicentre or Lucigen electrocompetent cells), along with electroporation cuvettes (we use Bio-Rad Gene Pulser cuvettes, 0.2 cm electrode gap) on ice.

Mix 1-10 μl of dialyzed ligation with 25-50 μl electrocompetent cells using a wide-bore pipet tip. Be careful not to introduce air bubbles into the sample, and leave on ice for 5 min while you add 1 ml SOC into culture tubes.

Transfer the cells and ligation mix into an electrocuvette. With one hand have a Pasteur pipet containing the 1 ml SOC, while you then electroporate the sample with your other hand, with the electroporator set at 25 μF, 200 Ohms, and 2.5 volts per sec. Immediately add the 1 ml SOC to the cuvette after electroporation, mix gently a few times, and transfer the cell suspension into the culture tube.

Incubate the cell suspension at 37° C. for 30 min.

Plate 100-200 μl of cell suspension onto LB agar containing appropriate antibiotic selection (12.5 μg ml⁻¹ Cm) and blue/white indicators (X-Gal and IPTG).

Incubate the agar plate overnight at 37° C. to recover colonies.

Alternatively, Sau3A enzyme may be added with buffer absent the MgCl₂, and then the MgCl₂ added to the tubes at different concentrations to initiate restriction digestion. The partially restriction-digested genomic DNA may be elctroeluted from the agarose gel, following published protocols (Osoegawa, K, Woon, P Y, Zhao, B, Frengen, E, Tateno, M, Catanese, J J, DeJong, P (1998) An improved approach for construction of bacterial artificial chromosome libraries. Genomics 52:1-8).

We run the samples on a normal agarose gel, rather than a pulsed field gel, since the standard agarose gel compresses the HMW DNA into a small region of the gel, making quantification easier. Given sufficient DNA, it is also a good idea to run 5-10 μl of sample on a pulsed field agarose gel, to evaluate molecular size.

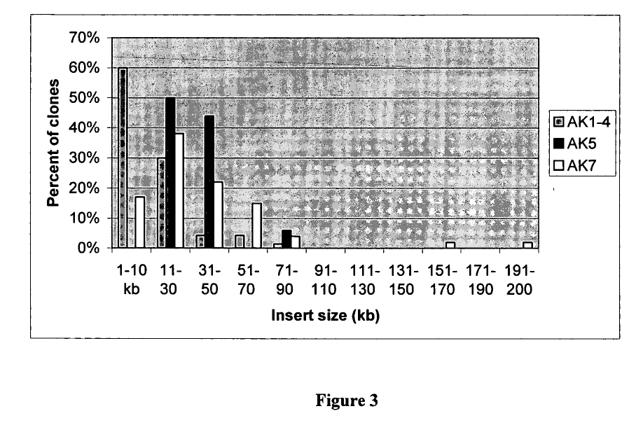

We recovered a small number of clones from WMARS and HARS HMW DNA (<1000 from both ligations) with an average insert size of 50 kb, and from BCEF soil several libraries were constructed, with insert sizes ranging from 13 kb (direct extraction; n=46,100), to 23 kb (indirect extraction, no size-selection; n=1400), to 47 kb (indirect extraction, 100 kb+size-selection n=2300) (FIG. 3). These results indicate that the recovery of microbial cells from soil has significant advantages in the purity and size of genomic DNA that can be achieved. Furthermore, the resultant HMW DNA may be cloned into a BAC vector after partial restriction digestion with Sau3A. Our ability to recover HMW DNA from three soils with high clay, silt, or sand contents, respectively, suggests that this methodology may be adopted by researchers working with many different soil and sediment types.

The foregoing description and examples have been set forth merely to illustrate the invention and are not intended to be limiting. All references cited herein are incorporated in their entireties by reference. Since modifications of the disclosed embodiments incorporating the spirit and substance of the invention may occur to persons skilled in the art, the invention should be construed broadly to include all variations falling within the scope of the appended claims and equivalents thereof.

1. A method for isolating polynucleotide molecules from microbial cells in an environmental sample, the method comprising:
(1) preparing a suspension of the environmental sample,
(2) optionally dispersing the cells from each other and from other components in the sample,
(3) purifying the dispersed cells via discontinuous gradient centrifugation wherein the cells are collected in an interface of the gradient,
(4) embedding the cells in agarose gel, to produce agarose gel blocks containing the cells, and
(5) lysing the cells within the agarose gel blocks, thereby releasing high molecular weight polynucleotide molecules from the cells.

2. The method of claim 1, wherein the environmental sample is selected from the group consisting of soil, marine sediment, fresh water, sea water, animal organs, animal part, plant rhizosphere, microbial mats, mine deposit and sulfur pools.

3. The method of claim 1, wherein in step (2) the suspension of the environmental sample is subject to a low speed blender homogenization.

4. The method according to claim 1, wherein in step (2) the cells are dissociated from particles in the environmental sample by treatment with a dissociation buffer comprising sodium deoxycholate, polyethylene glycol, and an anion exchange resin.

5. A method according to claim 1, wherein the discontinuous gradient comprises an aqueous phase and a Nycodenz phase.

6. The method according to claim 5, further comprising incorporating sodium metaphosphate in the suspension of the environmental sample, to increase yield of polynucleotide recovered.

7. The method of claim 1, wherein the purifying step (3) is repeated one or more times.

8. A method for cloning high molecular weight polynucleotide molecules from uncultured microbial cells in an environmental sample, comprising isolating said polynucleotide molecules according to claim 1, and further:
(1) incubating the gel blocks with at least a suitable restriction endonuclease to partially digest the polynucleotide molecules,
(2) separating the digested polynucleotide molecules by pulse field gel electrophoresis, to recover fragments of polynucleotide molecules with size of at least 50 kb,
(3) optionally removing agarose gel,
(4) ligating recovered fragments of polynucleotide molecules to a suitable cloning vector, and
(5) introducing into a suitable host cell with the cloning vector containing the fragments of the high molecular weight polynucleotide molecules.

9. The method according to claim 8, wherein the introducing in step (5) is transformation or transduction, resulting in a transformed cell comprising at least one said vector, further comprising recovering a transformed cell comprising at least one vector which comprises an insert polynucleotide molecule having a molecular size of at least about 50 kb.

10. The method of claim 9, further comprising recovering a transformed cell comprising at least one vector which comprises an insert polynucleotide molecule having a molecular size of at least about 50 kb.

11. The method according to claim 10, wherein the molecular size of the insert polynucleotide is at least about 75 kb.

12. The method according to claim 10, wherein the molecular size of the insert polynucleotide is at least about 100 kb.

13. The method according to claim 10, wherein the molecular size of the insert polynucleotide is at least about 150 kb.

14. The method according to claim 10, wherein the molecular size of the insert polynucleotide is at least about 200 kb.

15. A library comprising transformed cells recovered according to the method of claim 10.

16. The method according to claim 8, wherein in step (2), the gel blocks are incubated with a restriction enzyme at a temperature that inhibits non-specific nuclease activity.

17. The method according to claim 16, wherein the temperature is about 4° C.

18. The method of claim 8, wherein the cloning vector is a BAC vector.

19. The method of claim 8, wherein in step (2), the digested polynucleotide molecules are electro eluted in a dialysis membrane bag.

20. The method of claim 19, wherein the polynucleotide eluded from the gel blocks in the dialysis bag is further purified and concentrated.
